(19) United States
(12) Patent Application Publication
(10) Pub. No.: US 2022/0281830 A1
(43) Pub. Date: Sep. 8, 2022

(54) SALT

(71) Applicant: NIPPON SHINYAKU CO., LTD., Kyoto-shi, Kyoto (JP)

(72) Inventors: Yasushi KOKUBO, Kyoto-shi, Kyoto (JP); Toshitaka YAMAMOTO, Odawara-shi, Kanagawa (JP); Koji NAKAMICHI, Kyoto-shi, Kyoto (JP); Domenico CROCCO, Craigavon (GB)

(73) Assignee: NIPPON SHINYAKU CO., LTD., Kyoto-shi, Kyoto (JP)

(21) Appl. No.: 17/635,710

(22) PCT Filed: Aug. 19, 2020
(86) PCT No.: PCT/JP2020/031204

§ 371 Date: Feb. 16, 2022

(30) Foreign Application Priority Data
Aug. 19, 2019 (JP) 2019-149945

(51) Int. Cl. C07D 241/20; C07D 241/28

(52) U.S. Cl. CPC C07D 241/20; C07D 241/28; C07B 2200/13

(57) ABSTRACT

The present invention relates to a novel salt of 2-(4-(5,6-diphenylpyrazin-2-yl)(isopropyl)amino)butoxy)acetic acid (hereinafter referred to as “Compound B”) and a crystal of the salt thereof.

### TECHNICAL FIELD

The present invention relates to a novel salt of 2-(4-((5,6-diphenylpyrazin-2-yl)(isopropyl)amino)butoxy)acetic acid (hereinafter referred to as “Compound B”) and a crystal of the salt thereof.

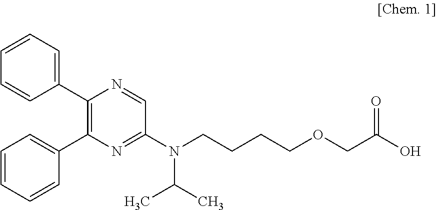 [SMILES: CC(C)N(CCCCOCC(=O)O)c1cnc(-c2ccccc2)c(-c2ccccc2)n1]

### BACKGROUND ART

A pharmaceutical product is required to maintain its quality over a long period of time even under various conditions of distribution, storage, etc. Therefore, a compound to serve as an active ingredient is required to have high physicochemical stability. Due to this, as an active ingredient of a pharmaceutical product, a salt and/or a crystal form which may be expected to have high stability is adopted.

In a process for screening a salt and/or a crystal of an active ingredient of a pharmaceutical product, not only is it difficult to find optimal conditions for obtaining the salt and/or the crystal, but also, even if the salt and/or the crystal is obtained, the solubility and the existence of polymorphism is often problematic. The problem is caused because there is a difference in physicochemical stability depending on the salt type and the crystal form.

However, it is impossible to predict the solubility of a salt and the existence of polymorphism or a stable salt and/or crystal form from the structure of a compound, and moreover, there exists a compound which cannot be crystallized in some cases, and it is necessary to variously study the conditions for forming a salt and/or a crystal for each compound.

Compound B is known to have an excellent PGI2 receptor agonistic effect and show various medicinal effects such as a platelet aggregation inhibitory effect, a vasodilating effect, a bronchial smooth muscle dilating effect, a lipid deposition inhibitory effect, and a leukocyte activation inhibitory effect (see, for example, PTL 1 to PTL 6). However, the current situation is that it is not known whether or not a salt and/or a crystal can be formed, much less whether or not polymorphism exists, and it is an important object to acquire an optimal salt and/or crystal for development thereof as a pharmaceutical product.

### CITATION LIST

### Patent Literature

[PTL 1] WO 2002/088084

[PTL 2] WO 2009/157396

[PTL 3] WO 2009/107736

[PTL 4] WO 2009/154246

[PTL 5] WO 2009/157397

[PTL 6] WO 2009/157398

[PTL 7] US 2014/0221397

[PTL 8] US 2011/0178103

[PTL 9] US 2011/0015211

[PTL 10] US 2011/0118254

[PTL 11] US 2011/0105518

### Non Patent Literature

[NPL 1] Hepatology, 2007, Vol. 45, No. 1, pp. 159-169

[NPL 2] PubMed: Nihon Yakurigaku Zasshi, 2001, February, 117(2), pp. 123-130, Abstract

[NPL 3] International Angiology, 29, Suppl. 1 to No. 2, pp. 49-54, 2010

[NPL 4] Japanese Journal of Clinical Immunology, Vol. 16, No. 5, pp. 409-414, 1993

[NPL 5] Japanese Journal of Thrombosis and Hemostasis, Vol. 1, No. 2, pp. 94-105, 1990, Abstract

[NPL 6] The Journal of Rheumatology, Vol. 36, No. 10, pp. 2244-2249, 2009

[NPL 7] The Japanese Journal of Pharmacology, Vol. 43, No. 1, pp. 81-90, 1987

[NPL 8] British Heart Journal, Vol. 53, No. 2, pp. 173-179, 1985

[NPL 9] The Lancet, 1, 4880, pt 1, pp. 569-572, 1981

[NPL 10] European Journal of Pharmacology, 449, pp. 167-176, 2002

[NPL 11] The Journal of Clinical Investigation, 117, pp. 464-72, 2007

[NPL 12] American Journal of Physiology Lung Cellular and Molecular Physiology, 296: L648-L656 2009

### SUMMARY OF INVENTION

### Technical Problem

An object of the present invention is to provide a salt and/or a crystal of Compound B having excellent physicochemical stability and pharmacokinetic property also to provide a pharmaceutical composition containing the salt and/or the crystal as an active ingredient.

### Solution to Problem

A method for producing Compound B is disclosed in Example 42 of PTL 1. When the present inventor produced Compound B according to the same procedure as the method disclosed in Example 42 of PTL 1, it was found that the free form is a crystal (hereinafter referred to as “form-III crystal”). However, it was found that the form-III crystal is thermodynamically unstable, and therefore, the present inventor made intensive studies in order to achieve the above object, and as a result, it was found that there exist a salt and/or a crystal, each of which is thermodynamically more stable and better pharmacokinetic property. Thus, the present invention was completed.

The present invention can include, for example, the following (1) to (22).

(1) Ammonium salt of 2[4-[(5,6-diphenylpyrazin-2-yl)-propan-2-ylamino]butoxy]acetic acid, or pharmaceutically acceptable hydrate or solvate thereof
(2) Arginate salt of 2-[4[(5,6-diphenylpyrazin-2-yl)-propan-2-ylamino]butoxy]acetic acid, or pharmaceutically acceptable hydrate or solvate thereof.
(3) Calcium salt of 2[4-[(5,6-diphenylpyrazin-2-yl)-propan-2-ylamino]butoxy]acetic acid, or pharmaceutically acceptable hydrate or solvate thereof.
(4) Choline salt of 2-[4-[(5,6-diphenylpyrazin-2-yl)-propan-2-ylamino]butoxy]acetic acid, or pharmaceutically acceptable hydrate or solvate thereof
(5) 1,2-Ethanedisulfonate salt of 2-[4-[(5,6-diphenylpyrazin-2-yl)-propan-2-ylamino]butoxy]acetic acid, or pharmaceutically acceptable hydrate or solvate thereof.
(6) Histidine salt of 2-[4-[(5,6-diphenylpyrazin-2-yl)-propan-2-ylamino]butoxy]acetic acid, or pharmaceutically acceptable hydrate or solvate thereof.
(7) Potassium salt of 2-[4-[(5,6-diphenylpyrazin-2-yl)-propan-2-ylamino]butoxy]acetic acid, or pharmaceutically acceptable hydrate or solvate thereof
(8) Sodium salt of 2-[4-[(5,6-diphenylpyrazin-2-yl)-propan-2-ylamino]butoxy]acetic acid, or pharmaceutically acceptable hydrate or solvate thereof.
(9) Tromethamine salt of 2-[4-[(5,6-diphenylpyrazin-2-yl)-propan-2-ylamino]butoxy]acetic acid, or pharmaceutically acceptable hydrate or solvate thereof.
(10) A crystal of the ammonium salt according to (1), showing diffraction peaks in its X-ray powder diffraction spectrum at least at the following angles of diffraction 2 θ: 8.4, 14.7, 15.2, 16.3 and 21.3 degree, preferably 2 θ: 8.4, 11.2, 14.7, 15.2, 16.3 and 21.3 degree, wherein the X-ray powder diffraction diagram is obtained by using Cu Kα radiation.
(11) A crystal of the L-arginine salt according to (2), showing diffraction peaks in its X-ray powder diffraction spectrum at least at the following angles of diffraction 2 θ: 5.5, 11.1, 19.3, 20.2 and 22.4 degree, preferably 2 θ: 5.5, 11.1, 19.3, 19.8, 20.2, 22.4 and 23.1 degree, wherein the X-ray powder diffraction diagram is obtained by using Cu Kα radiation.
(12) A crystal of the calcium salt according to (3), showing diffraction peaks in its X-ray powder diffraction spectrum at least at the following angles of diffraction 2 θ: 4.8, 8.7, 9.7, 15.2 and 18.5 degree, preferably 2 θ: 4.8, 8.7, 9.7, 11.1, 15.2, 16.0, 18.1, 18.5 and 23.4 degree, wherein the X-ray powder diffraction diagram is obtained by using Cu Kα radiation.
(13) A crystal of the choline salt according to (4), showing diffraction peaks in its X-ray powder diffraction spectrum at least at the following angles of diffraction 2 θ: 9.5, 10.4, 15.0, 17.8 and 21.5 degree, preferably 2 θ: 9.5, 10.4, 13.5, 15.0, 17.8, 18.6, 18.9, 20.5 and 21.5 degree, wherein the X-ray powder diffraction diagram is obtained by using Cu Kα radiation.
(14) A crystal of the 1,2-ethanedisulfonate salt according to (5), showing diffraction peaks in its

X-ray powder diffraction spectrum at least at the following angles of diffraction 2 θ: 6.8, 8.6, 19.4, 22.5 and 25.6 degree, preferably 2 θ: 6.8, 8.6, 10.1, 12.7, 16.2, 18.3, 19.4, 22.5 and 25.6, wherein the X-ray powder diffraction diagram is obtained by using Cu Kα radiation.

(15) A crystal of the L-histidine salt according to (6), showing diffraction peaks in its X-ray powder diffraction spectrum at least at the following angles of diffraction 2 θ: 9.4, 15.3, 18.9, 21.0 and 24.2 degree, preferably 2 θ: 9.4, 15.3, 18.9, 19.6, 21.0, 21.5, 24.2, 25.4, 30.2 and 30.9 degree, wherein the X-ray powder diffraction diagram is obtained by using Cu Kα radiation.
(16) A crystal of the potassium salt according to (7), showing diffraction peaks in its X-ray powder diffraction spectrum at least at the following angles of diffraction 2 θ: 5.9, 9.9, 18.7, 20.4 and 21.7 degree, preferably 2 θ: 5.9, 7.3, 9.3, 9.9, 10.4, 13.2, 18.7, 20.4, 21.7 and 22.5 degree, wherein the X-ray powder diffraction diagram is obtained by using Cu Kα radiation.
(17) A crystal of the potassium salt according to (7), showing diffraction peaks in its X-ray powder diffraction spectrum at least at the following angles of diffraction 4.0, 4.5, 8.2, 14.6 and 17.2 degree, preferably 2 θ: 4.0, 4.5, 8.2, 8.7, 14.6 and 17.2 degree, wherein the X-ray powder diffraction diagram is obtained by using Cu Kα radiation.
(18) A crystal of the sodium salt according to (8), showing diffraction peaks in its X-ray powder diffraction spectrum at least at the following angles of diffraction 2 θ: 5.9, 9.9, 10.4, 18.6 and 20.4 degree preferably 2 θ: 5.9, 7.2, 9.9, 10.4, 13.1, 18.6, 20.4, 21.6 and 22.5 degree, wherein the X-ray powder diffraction diagram is obtained by using Cu Kα radiation.
(19) A crystal of the sodium salt according to (8), showing diffraction peaks in its X-ray powder diffraction spectrum at least at the following angles of diffraction 2 θ: 3.8, 7.9, 10.3, 19.8 and 20.7 degree, preferably 2 θ: 3.8, 7.9, 9.4, 9.9, 10.3, 18.0, 19.8 and 20.7 degree, wherein the X-ray powder diffraction diagram is obtained by using Cu Kα radiation.
(20) A crystal of the tromethamine salt according to (9), showing diffraction peaks in its X-ray powder diffraction spectrum at least at the following angles of diffraction 2 θ: 4.0, 7.2, 15.5, 17.8 and 20.2 degree, preferably 2 θ: 4.0, 7.2, 8.0, 10.6, 15.5, 17.5, 17.8, 18.5 and 20.2 degree, wherein the X-ray powder diffraction diagram is obtained by using Cu Kα radiation.
(21) A crystal of the tromethamine salt according to (9), showing diffraction peaks in its X-ray powder diffraction spectrum at least at the following angles of diffraction 2 θ=3.5, 10.4, 15.9, 17.1 and 20.6 degree, preferably 2 θ: 3.5, 10.4, 15.9, 17.1, 17.6, 18.3, 19.9, 20.6, 21.9 and 24.0 degree, wherein the X-ray powder diffraction diagram is obtained by using Cu Kα radiation.
(22) A pharmaceutical composition containing the salt or the crystal according to any one of (1) to (21) as an active ingredient (hereinafter referred to as “pharmaceutical composition of the present invention”).

When specifying a diffraction angle (20) for a diffraction peak in Examples and the claims of the present invention, it should be understood that an obtained value is within the range of the value ±0.2°, preferably within the range of the value ±0.1°.

### BRIEF DESCRIPTION OF DRAWINGS

FIG. 1 shows a powder X-ray diffraction spectrum chart of Ammonium salt (Pattern A). The vertical axis represents a peak intensity (cps) and the horizontal axis represents a diffraction angle (2 θ[°]).

FIG. 2 shows a powder X-ray diffraction spectrum chart of Arginine salt (Pattern 1). The vertical axis represents a peak intensity (cps) and the horizontal axis represents a diffraction angle (2 θ[°]).

FIG. 3 shows a powder X-ray diffraction spectrum chart of L-Histidine salt (Pattern 1). The vertical axis represents a peak intensity (cps) and the horizontal axis represents a diffraction angle (2 θ[°]).

FIG. 4 shows a powder X-ray diffraction spectrum chart of Sodium salt Pattern 1. The vertical axis represents a peak intensity (cps) and the horizontal axis represents a diffraction angle (2 θ[°]).

FIG. 5 shows a powder X-ray diffraction spectrum chart of Tromethamine salt Pattern 1. The vertical axis represents a peak intensity (cps) and the horizontal axis represents a diffraction angle (2 θ[°]).

FIG. 6 shows a powder X-ray diffraction spectrum chart of Choline salt (Pattern 1). The vertical axis represents a peak intensity (cps) and the horizontal axis represents a diffraction angle (2 θ[°]).

FIG. 7 shows a powder X-ray diffraction spectrum chart of Potassium salt Pattern 2. The vertical axis represents a peak intensity (cps) and the horizontal axis represents a diffraction angle (2 θ[°]).

FIG. 8 shows a powder X-ray diffraction spectrum chart of Calcium salt (Pattern 1). The vertical axis represents a peak intensity (cps) and the horizontal axis represents a diffraction angle (2 θ[°]).

FIG. 9 shows a powder X-ray diffraction spectrum chart of 1,2-Ethanedisulfonate salt (Pattern A). The vertical axis represents a peak intensity (cps) and the horizontal axis represents a diffraction angle (2 θ[°]).

FIG. 10 shows a powder X-ray diffraction spectrum chart of Potassium salt Pattern 1. The vertical axis represents a peak intensity (cps) and the horizontal axis represents a diffraction angle (2 θ[°]).

FIG. 11 shows a powder X-ray diffraction spectrum chart of Sodium salt Pattern 2. The vertical axis represents a peak intensity (cps) and the horizontal axis represents a diffraction angle (2 θ[°]).

FIG. 12 shows a powder X-ray diffraction spectrum chart of Tromethamine salt Pattern 2. The vertical axis represents a peak intensity (cps) and the horizontal axis represents a diffraction angle (20 θ[°]).

### DESCRIPTION OF EMBODIMENTS

### A. Salt of the Present Invention

The salt of the present invention can be obtained by, for example, the method described in the below-mentioned Examples.

### B. Crystal of the Present Invention

The crystal of a salt of the present invention can be obtained by, for example, the method described in the below-mentioned Examples.

### C. Medical Application Pharmaceutical Composition of the Present Invention

The Compound B according to the present invention has an excellent PGI2 receptor agonistic effect and shows various medicinal effects such as a platelet aggregation inhibitory effect, a vasodilating effect, a bronchial smooth muscle dilating effect, a lipid deposition inhibitory effect, and a leukocyte activation inhibitory effect (see, for example, PTL 1).

Therefore, the salt and/or crystal of the present invention, or the pharmaceutical composition of the present invention is useful as a preventive agent or a therapeutic agent for transient ischemic attack (TIA), diabetic neuropathy (see, for example, NPL 1), diabetic gangrene (see, for example, NPL 1), a peripheral circulatory disturbance [for example, chronic arterial occlusion (see, for example, NPL 2), intermittent claudication (see, for example, NPL 3), peripheral embolism, vibration syndrome, or Raynaud's disease] (see, for example, NPL 4 and NPL 5), a connective tissue disease [for example, systemic lupus erythematosus, scleroderma (see, for example, PTL 7 and NPL 6), a mixed connective tissue disease, or a vasculitic syndrome], reocclusion/restenosis after percutaneous transluminal coronary angioplasty (PTCA), arteriosclerosis, thrombosis (for example, acute-phase cerebral thrombosis or pulmonary embolism) (see, for example, NPL 5 and NPL 7), hypertension, pulmonary hypertension, an ischemic disease [for example, cerebral infarction or myocardial infarction (see, for example, NPL 8)], angina pectoris (for example, stable angina pectoris or unstable angina pectoris) (see, for example, NPL 9), glomerulonephritis (see, for example, NPL 10), diabetic nephropathy (see, for example, NPL 1), chronic renal failure (see, for example, PTL 8), allergy, bronchial asthma (see, for example, NPL 11), ulcer, pressure ulcer (bedsore), restenosis after coronary intervention such as atherectomy or stent implantation, thrombocytopenia by dialysis, a disease in which fibrogenesis in an organ or a tissue is involved [for example, a renal disease {for example, tubulointerstitial nephritis (see, for example, PTL 9)}, a respiratory disease {for example, interstitial pneumonia (for example, pulmonary fibrosis) (see, for example, PTL 9), a chronic obstructive pulmonary disease (see, for example, NPL 12)}, a digestive disease (for example, hepatocirrhosis, viral hepatitis, chronic pancreatitis, or scirrhous gastric cancer), a cardiovascular disease (for example, myocardial fibrosis), a bone or articular disease (for example, bone marrow fibrosis or rheumatoid arthritis), a skin disease (for example, postoperative cicatrix, burn cicatrix, keloid, or hypertrophic cicatrix), an obstetric disease (for example, uterine fibroid), a urinary disease (for example, prostatic hypertrophy), other diseases (for example, Alzheimer's disease, sclerosing peritonitis, type I diabetes, and postoperative organ adhesion)], erectile dysfunction (for example, diabetic erectile dysfunction, psychogenic erectile dysfunction, psychotic erectile dysfunction, erectile dysfunction due to chronic renal failure, erectile dysfunction after pelvic operation for resection of the prostate, or vascular erectile dysfunction associated with aging or arteriosclerosis), an inflammatory bowel disease (for example, ulcerative colitis, Crohn's disease, intestinal tuberculosis, ischemic colitis, or intestinal ulcer associated with Behcet disease) (see, for example, PTL 10), gastritis, gastric ulcer, an ischemic eye disease (for example, retinal artery occlusion, retinal vein occlusion, or ischemic optic neuropathy), sudden hearing loss, avascular necrosis of bone, an intestinal damage caused by administration of a non-steroidal anti-inflammatory agent (NSAID) (for example, diclofenac, meloxicam, oxaprozin, nabumetone, indomethacin, ibuprofen, ketoprofen, naproxen, or celecoxib) (there is no particular limitation as long as it is a damage occurring in, for example, the duodenum, small intestine, or large intestine, however, for example, a mucosal damage such as erosion or ulcer occurring in the duodenum, small intestine, or large intestine), or symptoms (for example, paralysis, dullness in sensory perception, pain, numbness, or a decrease in walking ability) associated with spinal canal stenosis (for example, cervical spinal canal stenosis, thoracic spinal canal stenosis, lumbar spinal canal stenosis, coexisting cervical and lumbar spinal stenosis, or sacral spinal stenosis) (see PTL 11).
In addition, the salt and/or crystal of the present invention or the pharmaceutical composition of the present invention is also useful as an accelerating agent for gene therapy or angiogenic therapy such as autologous bone marrow transplantation, or an accelerating agent for angiogenesis in restoration of peripheral artery or angiogenic therapy.

### D. Preparation

When the salt and/or the crystal of the present invention is administered as a pharmaceutical, t the salt and/or the crystal is administered as it is, or is contained in a pharmaceutically acceptable nontoxic inert carrier in an amount within the range of, for example, 0.1% to 99.5%, preferably within the range of 0.5% to 90%.

Examples of the carrier include solid, semi-solid, or liquid diluents, fillers, and other auxiliary agents for pharmaceutical formulation. Among these, one type or two or more types can be used.

The pharmaceutical composition of the present invention may be in any form of preparations for oral administration such as a powder, a capsule, a tablet, a sugar-coated tablet, a granule, a powder preparation, a suspension, a liquid, a syrup, an elixir, and a troche, and parenteral preparations such as an injection, a suppository in a solid or liquid dosage unit, and an inhalation. It may be in the form of a sustained release preparation. Among these, particularly, preparations for oral administration such as a tablet are preferred.

The powder can be produced by grinding the salt and/or the crystal of the present invention to an appropriate fineness.

The powder preparation can be produced by grinding the salt and/or the crystal of the present invention to an appropriate fineness, and then mixing the ground salt and/or the crystal with a similarly ground pharmaceutical carrier, for example, an edible carbohydrate such as starch or mannitol. A flavor, a preservative, a dispersant, a colorant, a perfume, or the like can be arbitrarily added thereto.

The capsule can be produced by firstly filling a powder or a powder preparation formed into a powdery shape as described above or a granulated material as will be described in the section on the tablet in, for example, a capsule shell such as a gelatin capsule. Further, the capsule can be produced by mixing a lubricant or a fluidizing agent such as colloidal silica, talc, magnesium stearate, calcium stearate, or solid polyethylene glycol with a powder or a powder preparation formed into a powdery shape, and thereafter performing a filling operation. It is possible to improve the effectiveness of the pharmaceutical when the capsule is taken if a disintegrating agent or a solubilizing agent such as carboxymethyl cellulose, carboxymethyl cellulose calcium, low-substituted hydroxypropyl cellulose, croscarmellose sodium, carboxymethyl starch sodium, calcium carbonate, or sodium carbonate is added thereto.
Further, it is also possible to form a soft capsule by suspending and dispersing the fine powder of the salt and/or the crystal of the present invention in a vegetable oil, polyethylene glycol, glycerin, or a surfactant, and wrapping the resulting material with a gelatin sheet.
The tablet can be produced by adding an excipient to the powdered salt and/or the crystal of the present invention to prepare a powder mixture, granulating or slagging the powder mixture, and then adding a disintegrating agent or a lubricant thereto, followed by tableting.
The powder mixture can be prepared by mixing the suitably powdered salt and/or the crystal of the present invention with a diluent or a base. If necessary, it is possible to add a binder (for example, carboxymethyl cellulose sodium, methyl cellulose, hydroxypropylmethyl cellulose, gelatin, polyvinylpyrrolidone, or polyvinyl alcohol), a dissolution retarding agent (for example, paraffin), a reabsorbing agent (for example, a quaternary salt), an adsorbent (for example, bentonite or kaolin), or the like thereto.
The granule can be produced by firstly wetting the powder mixture with a binder, for example, a syrup, a starch paste, gum Arabic, a cellulose solution, or a polymeric substance solution, stirring and mixing the wet mixture, and then, drying and crushing the mixture. In place of the granulation of the powder in this manner, it is also possible to form the granule by firstly subjecting the powder to a tableting machine, and thereafter crushing the obtained slag in an incomplete shape. By adding stearic acid, a stearate salt, talc, a mineral oil, or the like as a lubricant to the thus produced granule, the granules can be prevented from adhering to each other.
Further, the tablet can also be produced by mixing the salt and/or the crystal of the present invention with a fluid inert carrier, and thereafter directly tableting the resulting mixture without undergoing a granulation or slagging step as described above.
The thus produced tablet can be subjected to film coating or sugar coating. It is also possible to use a transparent or semi-transparent protective coating film made of a shellac sealing coating film, a coating film made of a sugar or a polymeric material, or a polished coating film made of a wax.
Another preparation for oral administration, for example, a liquid, a syrup, a troche, or an elixir can also be formulated into a dosage unit form such that a predetermined amount thereof contains a predetermined amount of the salt and/or the crystal of the present invention.
The syrup can be produced by dissolving the salt and/or the crystal of the present invention in an appropriate aqueous flavor solution. The elixir can be produced using a non-toxic alcohol carrier.
The suspension can be produced by dispersing the salt and/or the crystal of the present invention in a non-toxic carrier. If necessary, it is possible to add a solubilizing agent or an emulsifier (for example, an ethoxylated isostearyl alcohol or a polyoxyethylene sorbitol ester), a preservative, a flavor-imparting agent (for example, peppermint oil or saccharine), or the like thereto.
If necessary, the dosage unit formulation for oral administration may be microencapsulated. It is also possible to extend the duration of action or achieve sustained release by coating the formulation or embedding the formulation in a polymer, a wax, or the like.
The preparation for parenteral administration may be in a liquid dosage unit form for intramuscular or intravenous injection, for example, in the form of a solution or a suspension. The preparation for parenteral administration can be produced by suspending or dissolving a predetermined amount of the salt and/or the crystal of the present invention in a non-toxic liquid carrier meeting the purpose of injection, for example, an aqueous or oily medium, and then sterilizing the suspension or solution. It is also possible to add a stabilizing agent, a preservative, an emulsifier, or the like thereto.
The suppository can be produced by dissolving or suspending the salt and/or the crystal of the present invention in a solid which has a low melting point and is soluble or insoluble in water, for example, polyethylene glycol, cacao butter, a semi-synthetic oil or fat [for example, Witepsol (registered trade mark)], a higher ester (for example, myristyl palmitate ester), or a mixture thereof.

The dose varies depending on the state of a patient such as body weight or age, the administration route, the nature and degree of a disease, or the like, however, the dose as the amount of the salt and/or the crystal of the present invention per day per adult is suitably within the range of 0.001 mg to 100 mg, preferably within the range of 0.01 mg to 10 mg.

In some cases, a dose not more than the above range may be sufficient, or on the other hand, a dose not less than the above range may be needed. Further, the preparation can be administered once to several times a day or can be administered with an interval of one to several days.

### EXAMPLES

The present invention is described in more detail with reference to Examples and Test Examples given below; however, the present invention should not be limited whatsoever to these Examples. For the powder X-ray diffractometry, Panalytical Xpert Pro (target: Cu, voltage: 45 kV, current: 40 mA, scan speed: 0.2, 0.8, 1.7, or 3.4 degrees/min) was used.

### Example 1: Ammonium Salt (Pattern A)

### Example 2: Arginine Salt (Pattern 1)

### Example 3: L-Histidine Salt (Pattern 1)

### Example 4: Sodium salt (Pattern 1)

### Example 5: Tromethamine Salt (Pattern 1)

Salts of MRE-269, i e , Ammonium salt (Pattern A), Arginine salt (Pattern 1), L-Histidine salt (Pattern 1), Sodium salt (Pattern 1), and Tromethamine salt (Pattern 1) were prepared as follows.

### Method A:

### Preparation of MRE-269 Stock Solution

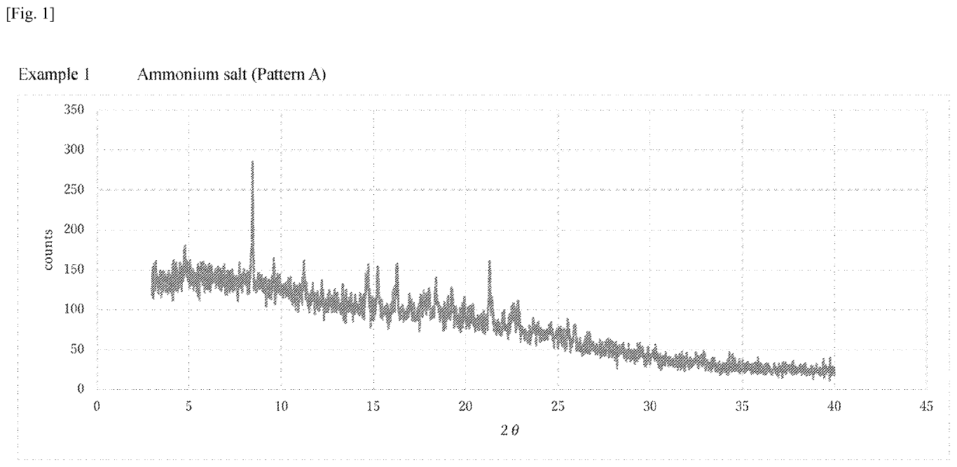

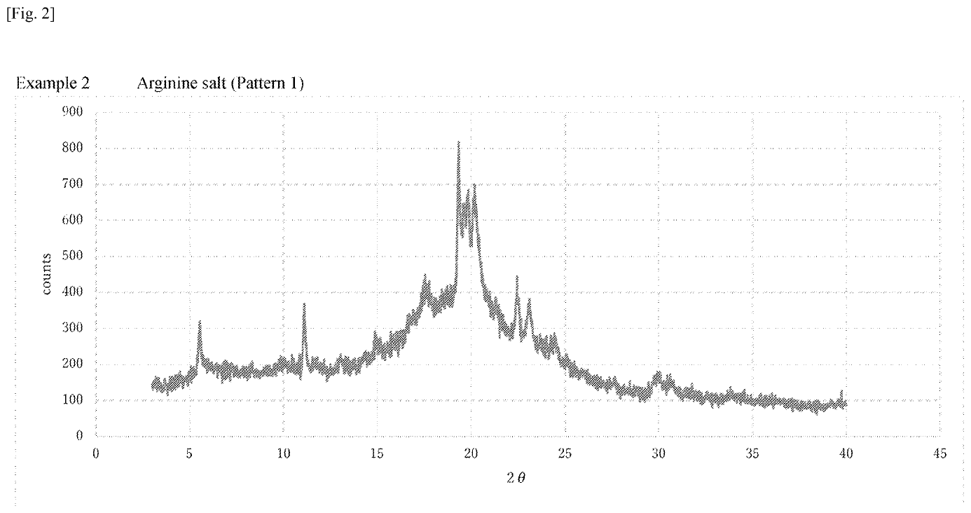

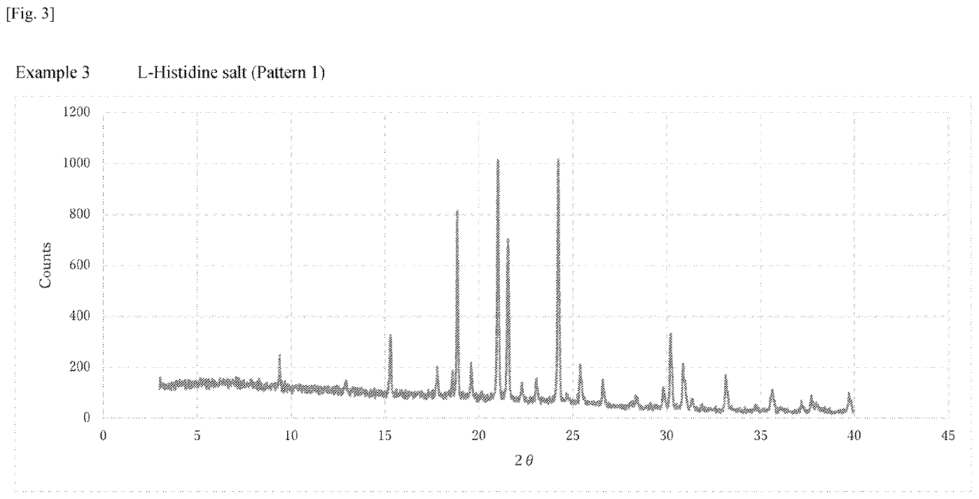

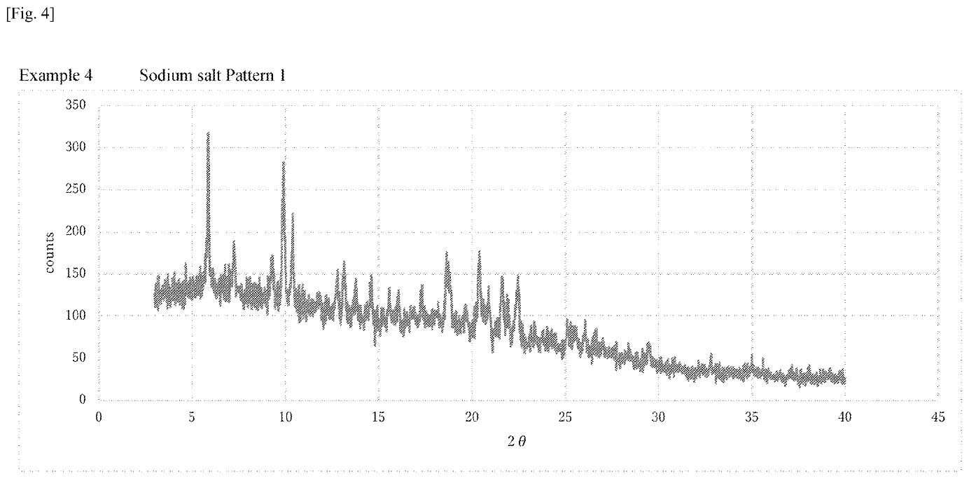

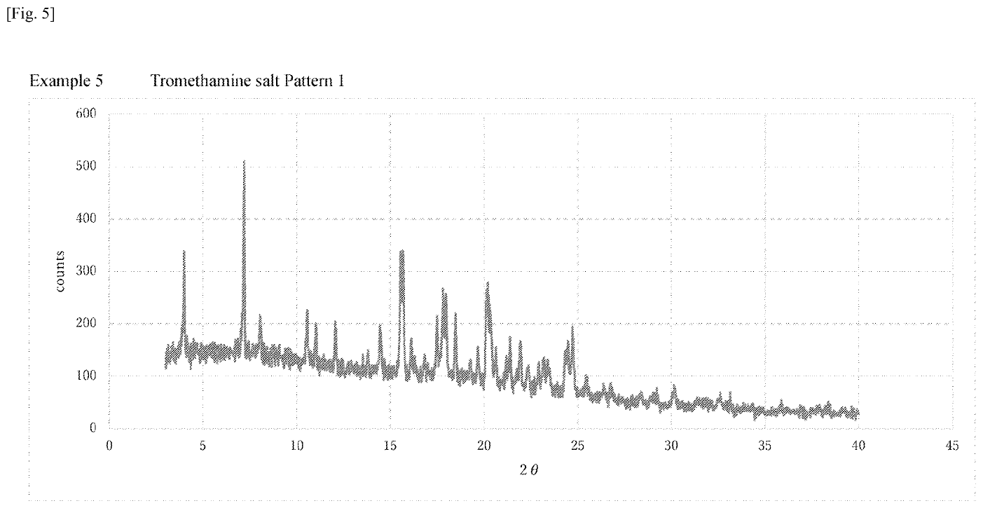

MRE-269 (804.4 mg) was weighed into a 20 mL volumetric flask and dissolved in THF (20 mL) at 40° C. for 2 hours. The MRE-269 aliquot (40 mg)=0.995 mL, 0.096 M was used for each experiment.
The MRE-269 stock solution (0.995 mL) prepared as described in Method A was added to a HPLC vial and heated at 40° C. for 1 hour. The corresponding co-former was charged to a vial at ambient temperature. The pre-warmed MRE-269 stock solution was charged to the vial containing the co-former and stirred for 24 hours at 40° C. The solution was allowed to cool to room temperature for 72 hours and resulting solids were isolated by centrifuge filtration and air dried for 5 minutes prior to analysis by XRPD (FIGS. 1 to 5).

### Example 6: Choline Salt (Pattern 1)

### Method B:

### Preparation of MRE-269 Stock Solution

MRE-269 (805 mg) was weighed into a 20 mL volumetric flask and dissolved in THF (20 mL) at 40° C. for 2 hours. The MRE-269 aliquot (40 mg)=0.993 mL, 0.096 M was used for the experiment.

### Preparation of the Co-former Stock Solution

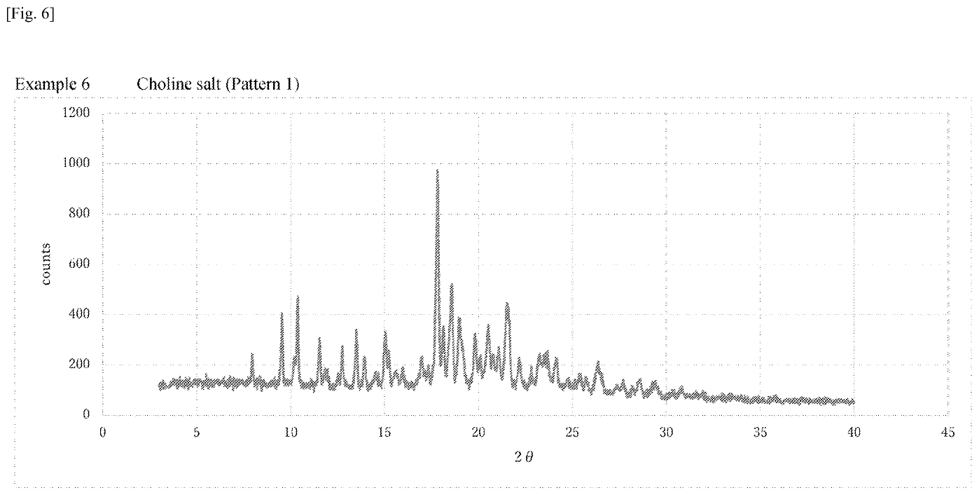

Co-former stock solutions were prepared at 0.1 M concentration.
MRE-269 stock solution and the co-former stock solution were prepared as described in Method B.
The MRE-269 stock solution (0.993 mL) was added to a HPLC vial and heated at 40° C. for 1 hour. The co-former stock solution was added (1 mol. eq.), stirred at 40° C. for 1 hour, allowed to cool to room temperature for up to 72 hours. The solution was evaporated using a nitrogen flow at ambient temperature. The gel was isolated and then was triturated in acetone (100 μL) for 7 days and the acetone was evaporated. The gel was slurried in MTBE. The MTBE was evaporated and the gel was dried, under vacuum, in a desiccator for up to 7 days. The solids were analysed by XRPD (FIG. 6).

### Example 7: Potassium Salt Pattern 2

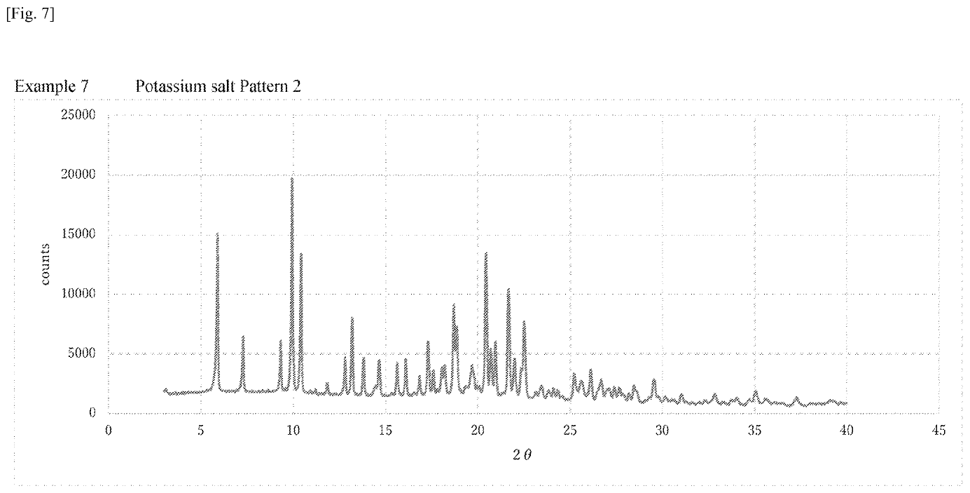

MRE-269 (400.2 mg), KOH (53.20 mg, 1 molar eq.) and THF (4 mL) were added to a vial and mixed at 40° C. for 24 hours. The solution was allowed to cool to room temperature and the solids were isolated by vacuum filtration using a Buchner funnel and dried, under vacuum, for 5 minutes. The product was dried at ambient temperature in the fume hood for 12 hours prior to analysis by XRPD (FIG. 7). MRE-269 potassium salt was isolated (470 mg, 90% yield).

### Example 8: Calcium Salt (Pattern 1)

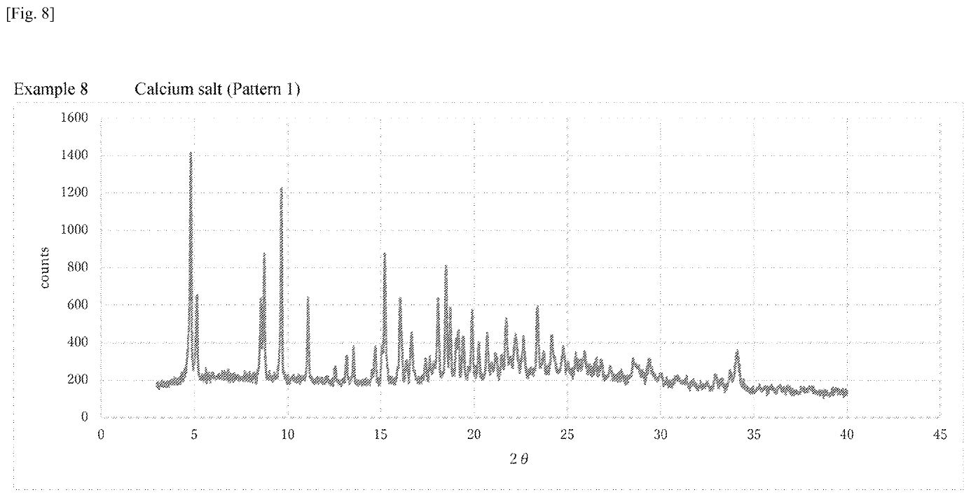

MRE-269 (5.99 g), calcium hydroxide (1.06 g, 1 molar eq.) and EtOH/water (1:1 v/v, 150 mL) were added to a vial. This was mixed at 50° C. for 2 days and the solution was allowed to cool to room temperature. The solids were isolated by vacuum filtration and air dried for 5 minutes, then dried at ambient for 12 hours prior to analysis by XRPD (FIG. 8). MRE-269 calcium salt (6.56 g, 93% yield) was isolated.

### Example 9: 1,2-Ethanedisulfonate Salt (Pattern A)

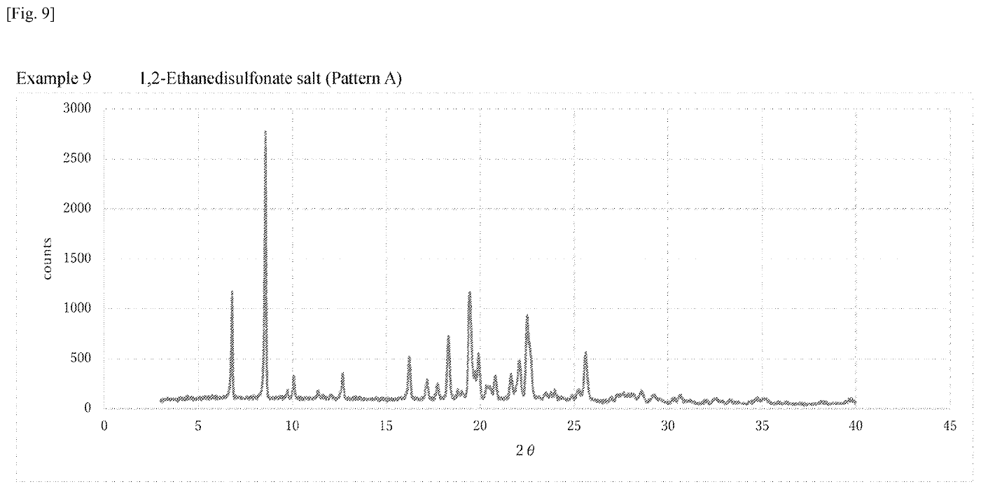

MRE-269 (6.01 g), 1,2-ethanedisulfonic acid (3.23 g, 1 molar eq.) and THF (60 mL) were added to a vial and mixed at 40° C. for 24 hours. The solution was allowed to cool to room temperature and the solids were isolated by vacuum filtration using a Buchner funnel and dried, under vacuum, for 5 minutes. The product was dried at ambient in the fume-hood for 12 hours prior to analysis by XRPD (FIG. 9). MRE-269 1,2-ethanedisulfonate salt (7.56 g, 81% yield) was recovered. 1H NMR analysis conforms to the molecular structure, the ratio of MRE-269 to 1,2-ethanedisulfonate is 1:0.5.

### Example 10: Potassium Salt Pattern 1

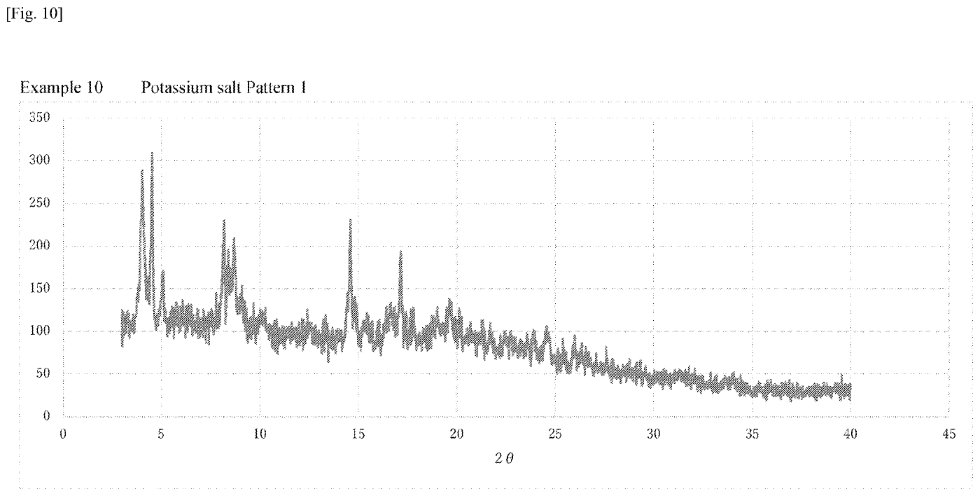

MRE-269 (6.00 g), KOH (0.84 g, 1 mol. eq.) and THF (60 mL) were added to a vial and mixed at 40° C. for 24 hours. The solution was allowed to cool to room temperature and an aliquot of the material was analysed by XRPD, it was obtained amorphous and MRE-269 potassium salt Pattern 1. The suspension was seeded with MRE-269 potassium salt Pattern 2. An aliquot was analysed after 24 hours, and MRE-269 salt Pattern 1 was isolated. The bulk material was isolated by vacuum filtration using a Buchner funnel and dried, under vacuum, for 5 minutes. The product was dried at RT in the fume hood for 12 hours prior to analysis by XRPD (FIG. 10). MRE-269 potassium salt Pattern 1 (5.88 g, 84% yield) was recovered.

### Example 11: Sodium Salt Pattern 2

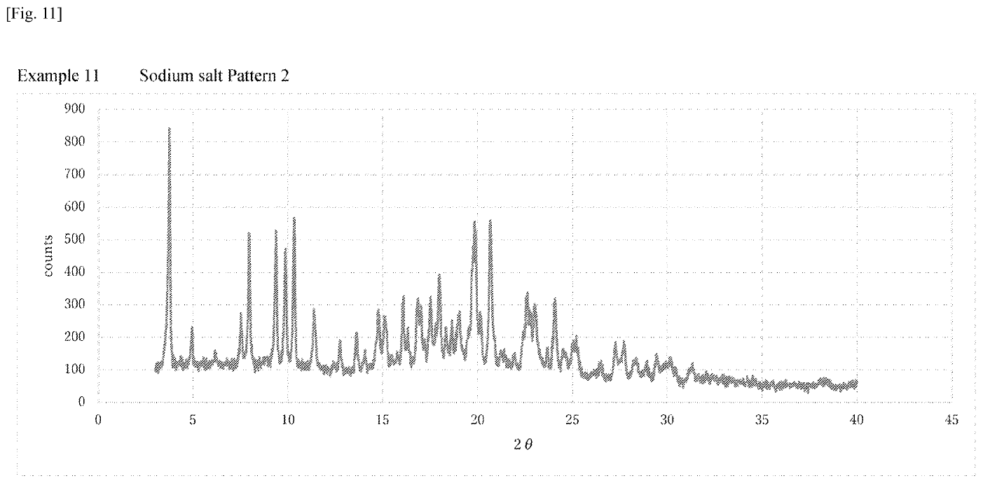

MRE-269 (9.01 g), NaOH (1.031 g, 1 molar eq.) and THF (90 mL) were added to a vial and mixed at 40° C. for 24 hours. A suspension was formed. THF (50 mL) was added to the suspension and the mixture was stirred at 40° C. for 24 hours. The suspension was allowed to cool to room temperature and the material was isolated by vacuum filtration using a Buchner funnel at ambient temperature. The material deliquesced. The material was washed with 3×5 mL aliquots of diethyl ether (Et2O) and a white solid formed. The solid was isolated by vacuum filtration using a Buchner funnel and dried, under vacuum, for 5 minutes. The product was dried in the fume hood for 12 hours prior to analysis by XRPD. MRE-269 sodium salt Pattern 2 (7 g, 70% yield) was recovered. XRPD analysis confirmed formation of MRE-269 sodium salt Pattern 2 (FIG. 11). The sodium content of the salt was determined using a Horiba Scientific LAQUAtw in compact water quality meter. The meter was calibrated at 150 and 2000 ppm. A solution of MRE-269 sodium salt (10 mg in 5 mL) in water was prepared and was added to the meter and the stoichiometry was 1:1.

### Example 12: Tromethamine Salt Pattern 2

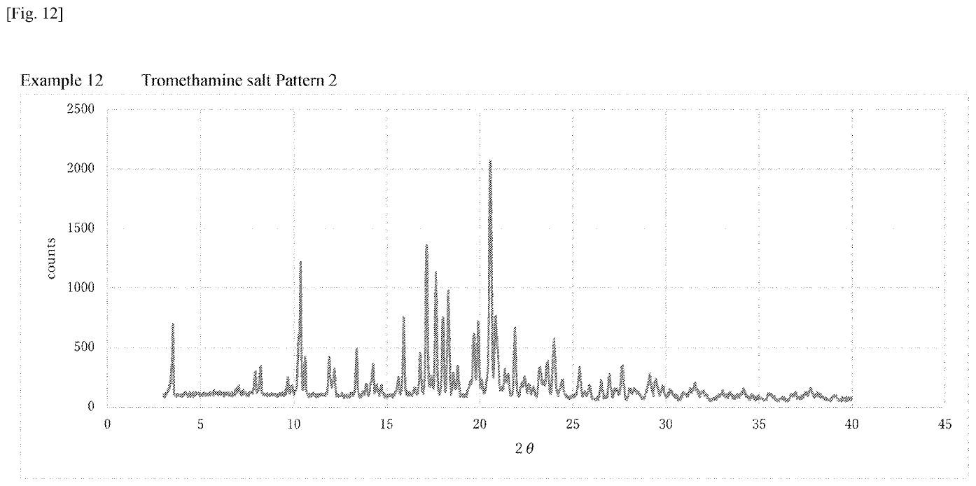

MRE-269 tromethamine salt Pattern 1 of Example 5 was stressed at 40° C./75% RH for one week to obtain tromethamine Pattern 2 material (seeds).
MRE-269 (6.00 g), tromethamine (1.737 g, 1 mol. eq.) and THF/water (3:1 v/v, 60 mL) were added to a vial and mixed at 40° C. for 24 hours. The solution was seeded with 200 mg of tromethamine Pattern 2 material (seeds dissolved). The solution was evaporated at 40° C. to 20 mL volume.
The solution was seeded with tromethamine salt Pattern 2 and Et2O (15 mL) was added. The solution was evaporated at ambient temperature for 48 hours to yield a pale yellow gel. The gel was triturated in acetone (75 mL) and stirred for 30 minutes to precipitate a solid which was collected by vacuum filtration.
The solid was washed with acetone (2×20 mL) and dried in the filter funnel for 10 minutes.
It was transferred to a crystallising basin and dried in the fume-hood for 2 hours. XRPD analysis (FIG. 12) was performed and MRE-269 tromethamine salt Pattern 2 material was recovered (6.0 g, 78% yield). The 1H NMR spectrum conforms to molecular structure and is consistent with formation of a mono-salt.

### Example 13: Solubility of MRE-269 Salts

The solubility of MRE-269 salts in water was determined and the results are shown in Table 1. The salts were stirred in water at 25 ° C. for ˜24 hours prior to filtration and HPLC analysis of the filtrate.

TABLE 1

| Salt | Solubility (mg/mL) |
|---|---|
| Calcium salt (Pattern 1) | 0.0021 |
| 1,2-Ethanedisulfonate salt (Pattern A) | <0.0002 |
| Sodium salt Pattern 2 | 29.9 |
| Tromethamine salt Pattern 2 | 119 |
| Potassium salt Pattern 2 | 1.2 |

### Rat In Vivo Study

MRE-269 and salts of 1,2-Ethanedisulfonate salt, Potassium salt, Tromethamine salt, Sodium salt and Calcium salt were encapsulated in HPMC capsule (for rodents; Qualicaps, Nara, Japan) and given orally to male Sprague-Dawley rat (Japan SLC, Sizuoka, Japan) at a dose level of 1 mg/capsule/body. Blood samples were collected from the jugular vein into heparinised tubes after oral dosing at 0.25, 0.5, 1, 2, 4, 6, 8, 10, and 24 hr post-dose. The tubes were centrifuged (16200×g, 4° C., 5 min) and the supernatant (0.2 ml) was transferred to a sample tube and stored at −20° C. in the refrigerator until analysis.

### Analytical Methods

The concentration of MRE-269 in plasma sample was determined by high performance liquid chromatography (HPLC) tandem mass spectrometry (LC-MS/MS). The plasma sample (20 μL) was mixed with 800 μL of acetonitrile containing internal standard compound (final concentration, 20 ng/mL), and centrifuged (16200×g, 4° C., 5 min) The resulting supernatants filtered using Ostro™ 96-well filter plates (Waters, Milford, Mass.). Samples of the filtered supernatants (1.0-2.5 μL) were subjected to LC-MS/MS. For LC-MS/MS, a Nexera HPLC system (Shimadzu, Kyoto, Japan) equipped with an InertSustain C18 column (2-μm particle size; 2.1 mm i.d.×50 mm; GL Science) connected to a TQ4500 or TQ5500 tandem mass spectrometer (Sciex, Framingham, Mass.) was used. The HPLC mobile phase was 0.1% formic acid (solvent A) and acetonitrile (solvent B). A gradient was run at 85% solvent B in 2.5 min at a flow rate of 0.4 mL/min and 40° C. Each analyte detected was quantified in positive ion multiple reaction monitoring mode by applying the following precursor-to-product transitions: MRE-269 m/z 420→260 and internal standard compound m/z 427→379 The analytical data were processed using Analyst 1.6.3 software (Sciex) for TQ4500 and Analyst 1.6.2 software (Sciex) for TQ5500.

### Pharmacokinetic Analysis

The pharmacokinetic parameters were calculated with Phoenix WinNonlin version 8.1.0 (Certara Princeton, NJ). Relative bioavailability (BA) to MRE-269 was calculated according to the following equation. Relative BA (%)=(AUC₀₋₂₄(each salt)/(AUC₀₋₂₄(MRE-269, mean)×100

TABLE 2
Result

| | | | | | | relative |
|---|---|---|---|---|---|---|
| | Cₘₐₓ | tₘₐₓ | t_{1/2} | AUC₀₋ₗₐₛₜ | AUC_{0-∞} | BA |
| | (ng/mL) | (hr) | (hr) | (ng · hr/mL) | (ng · hr/mL) | (%) |
| MRE-269 | 475 ± 142 | 6.50 ± 3.00 | NC | 5620 ± 1920 | NC | — |
| 1,2-Ethanedisulfonate | 1190 ± 1190 | 3.10 ± 2.30 | 3.99 ± 2.01 | 6540 ± 2930 | 6730 ± 3020 | 116 ± 52 |
| salt | | | | | | |
| Potassium salt | 937 ± 402 | 3.25 ± 3.20 | 8.96 ± 8.00 | 6130 ± 1520 | 8670 ± 2100 | 109 ± 27 |
| Tromethamine salt | 949 ± 555 | 4.00 ± 3.94 | 3.48 ± 0.43 | 7170 ± 2320 | 6520 ± 1890 | 128 ± 41 |
| Sodium salt | 578 ± 131 | 7.50 ± 1.91 | NC | 6300 ± 2160 | NC | 112 ± 38 |
| Calcium salt | 526 ± 234 | 7.50 ± 3.79 | NC | 5110 ± 1080 | NC | 90.9 ± 19.2 |

NC: Not Calculated

1. Ammonium salt of 2[4-[(5,6-diphenylpyrazin-2-yl)-propan-2-ylamino]butoxy]acetic acid, or a pharmaceutically acceptable hydrate or solvate thereof.

2. Arginate salt of 2[4-[(5,6-diphenylpyrazin-2-yl)-propan-2-ylamino]butoxy]acetic acid, or a pharmaceutically acceptable hydrate or solvate thereof.

3. Calcium salt of 2[4-[(5,6-diphenylpyrazin-2-yl)-propan-2-ylamino]butoxy]acetic acid, or a pharmaceutically acceptable hydrate or solvate thereof.

4. Choline salt of 2[4-[(5,6-diphenylpyrazin-2-yl)-propan-2-ylamino]butoxy]acetic acid, or a pharmaceutically acceptable hydrate or solvate thereof.

5. 1,2-Ethanedisulfonate salt of 2[4-[(5,6-diphenylpyrazin-2-yl)-propan-2-ylamino]butoxy]acetic acid, or a pharmaceutically acceptable hydrate or solvate thereof.

6. Histidine salt of 2[4-[(5,6-diphenylpyrazin-2-yl)-propan-2-ylamino]butoxy]acetic acid, or a pharmaceutically acceptable hydrate or solvate thereof.

7. Potassium salt of 2[4-[(5,6-diphenylpyrazin-2-yl)-propan-2-ylamino]butoxy]acetic acid, or a pharmaceutically acceptable hydrate or solvate thereof.

8. Sodium salt of 2[4-[(5,6-diphenylpyrazin-2-yl)-propan-2-ylamino]butoxy]acetic acid, or a pharmaceutically acceptable hydrate or solvate thereof.

9. Tromethamine salt of 2[4-[(5,6-diphenylpyrazin-2-yl)-propan-2-ylamino]butoxy]acetic acid, or a pharmaceutically acceptable hydrate or solvate thereof.

10. A crystal of the ammonium salt according to claim 1, showing diffraction peaks in its X-ray powder diffraction spectrum at least at the following angles of diffraction 2 θ: 8.4°, 14.7°, 15.2°, 16.3° and 21.3°, wherein the X-ray powder diffraction spectrum is obtained by using Cu Kα radiation.

11. A crystal of the L-arginine salt according to claim 2, showing diffraction peaks in its X-ray powder diffraction spectrum at least at the following angles of diffraction 2 θ: 5.5°, 11.1°, 19.3°, 20.2° and 22.4°, wherein the X-ray powder diffraction spectrum is obtained by using Cu Kα radiation.

12. A crystal of the calcium salt according to claim 3, showing diffraction peaks in its X-ray powder diffraction spectrum at least at the following angles of diffraction 2 θ: 4.8°, 8.7°, 9.7°, 15.2° and 18.5°, wherein the X-ray powder diffraction spectrum is obtained by using Cu Kα radiation.

13. A crystal of the choline salt according to claim 4, showing diffraction peaks in its X-ray powder diffraction spectrum at least at the following angles of diffraction 2 θ: 9.5°, 10.4°, 15.0°, 17.8° and 21.5°, wherein the X-ray powder diffraction spectrum is obtained by using Cu Kα radiation.

14. A crystal of the 1,2-ethanedisulfonate salt according to claim 5, showing diffraction peaks in its X-ray powder diffraction spectrum at least at the following angles of diffraction 2 θ: 6.8°, 8.6°, 19.4°, 22.5° and 25.6°, wherein the X-ray powder diffraction spectrum is obtained by using Cu Kα radiation.

15. A crystal of the L-histidine salt according to claim 6, showing diffraction peaks in its X-ray powder diffraction spectrum at least at the following angles of diffraction 2 θ: 9.4°, 15.3°, 18.9°, 21.0° and 24.2°, wherein the X-ray powder diffraction spectrum is obtained by using Cu Kα radiation.

16. A crystal of the potassium salt according to claim 7, showing diffraction peaks in its X-ray powder diffraction spectrum at least at the following angles of diffraction 2 θ: 5.9°, 9.9°, 18.7°, 20.4° and 21.7°, wherein the X-ray powder diffraction spectrum is obtained by using Cu Kα radiation.

17. A crystal of the potassium salt according to claim 7, showing diffraction peaks in its X-ray powder diffraction spectrum at least at the following angles of diffraction 2 θ: 4.0°, 4.5°, 8.2°, 14.6° and 17.2°, wherein the X-ray powder diffraction spectrum is obtained by using Cu Kα radiation.

18. A crystal of the sodium salt according to claim 8, showing diffraction peaks in its X-ray powder diffraction spectrum at least at the following angles of diffraction 2 θ: 5.9°, 9.9°, 10.4°, 18.6° and 20.4°, wherein the X-ray powder diffraction spectrum is obtained by using Cu Kα radiation.

19. A crystal of the sodium salt according to claim 8, showing diffraction peaks in its X-ray powder diffraction spectrum at least at the following angles of diffraction 2 θ: 3.8°, 7.9°, 10.3°, 19.8° and 20.7°, wherein the X-ray powder diffraction spectrum is obtained by using Cu Kα radiation.

20. A crystal of the tromethamine salt according to claim 9, showing diffraction peaks in its X-ray powder diffraction spectrum at least at the following angles of diffraction 2 θ: 4.0°, 7.2°, 15.5°, 17.8° and 20.2°, wherein the X-ray powder diffraction spectrum is obtained by using Cu Kα radiation.

21. A crystal of the tromethamine salt according to claim 9, showing diffraction peaks in its X-ray powder diffraction spectrum at least at the following angles of diffraction 2 θ: 3.5°, 10.4°, 15.9°, 17.1° and 20.6°, wherein the X-ray powder diffraction spectrum is obtained by using Cu Kα radiation.

22. A pharmaceutical composition containing the salt or the crystal according to any one of claims 1 to 21 as an active ingredient.

23. (canceled)
